(19) United States
(12) Patent Application Publication
(10) Pub. No.: US 2014/0316278 A1
(43) Pub. Date: Oct. 23, 2014

(54) SYSTEM AND METHOD FOR SCALING A FLUID RESPONSIVENESS METRIC

(71) Applicant: COVIDIEN LP, MANSFIELD, MA (US)

(72) Inventors: Paul Stanley Addison, Edinburgh (GB); Rui Wang, Livingston (GB); Scott McGonigle, Edinburgh (GB); James N. Watson, Edinburgh (GB)

(73) Assignee: COVIDIEN LP, MANSFIELD, MA (US)

(21) Appl. No.: 14/259,812

(22) Filed: Apr. 23, 2014

Related U.S. Application Data
(60) Provisional application No. 61/815,098, filed on Apr. 23, 2013.
(60) Provisional application No. 61/814,900, filed on Apr. 23, 2013.
(60) Provisional application No. 61/815,882, filed on Apr. 25, 2013.

(51) Int. Cl. A61B 5/00

(52) U.S. Cl. CPC A61B 5/0082
USPC 600/476

(57) ABSTRACT

The present invention relates to physiological signal processing, and in particular to methods and systems for processing physiological signals to predict a fluid responsiveness of a patient. A medical monitor for monitoring a patient may include an input receiving a photoplethysmograph (PPG) signal representing light absorption by a patient's tissue, and a fluid responsiveness predictor (FRP) calculator programmed to calculate an FRP metric. The monitor also may include a memory storing a relationship between the FRP metric and a pulse pressure variation (PPV) metric. The FRP metric is calculated based on a respiratory variation of the PPG signal and based on the relationship.

### CROSS-REFERENCE TO RELATED APPLICATIONS

The present application relates to and claims priority benefits from U.S. Provisional Application No. 61/815,098, filed Apr. 23, 2013, and U.S. Provisional Application No. 61/814,900, filed Apr. 23, 2013, and U.S. Patent Application No. 61/815,882, filed Apr. 25, 2013, the contents of which are hereby expressly incorporated by reference.

### FIELD

The present invention relates to physiological signal processing, and in particular to methods and systems for processing physiological signals to predict a fluid responsiveness of a patient.

### BACKGROUND

Fluids are commonly delivered to a patient in order to improve the patient's hemodynamic status. Fluid is delivered with the expectation that it will increase the patient's cardiac preload, stroke volume, and cardiac output, resulting in improved oxygen delivery to the organs and tissue. Fluid delivery may also be referred to as volume expansion, fluid therapy, fluid challenge, or fluid loading.

However, improved hemodynamic status is not always achieved by fluid loading. Moreover, inappropriate fluid loading may worsen a patient's status, such as by causing hypovolemia to persist (potentially leading to inadequate organ perfusion), or by causing hypervolemia (potentially leading to peripheral or pulmonary edema).

Respiratory variation in the arterial blood pressure waveform is known to be a good predictor of a patient's response to fluid loading, or fluid responsiveness. Fluid responsiveness represents a prediction of whether such fluid loading will improve blood flow within the patient. Fluid responsiveness refers to the response of stroke volume or cardiac output to fluid administration. A patient is said to be fluid responsive if fluid loading does accomplish improved blood flow, such as by an improvement in cardiac output or stroke volume index by about 15% or more. In particular, the pulse pressure variation (PPV) parameter from the arterial blood pressure waveform has been shown to be a good predictor of fluid responsiveness. This parameter can be monitored while adding fluid incrementally, until the PPV value indicates that the patient's fluid responsiveness has decreased, and more fluids will not be beneficial to the patient. This treatment can be accomplished without needing to calculate blood volume or cardiac output directly. This approach, providing incremental therapy until a desired target or endpoint is reached, may be referred to as goal-directed therapy (GDT).

However, PPV is an invasive metric, requiring the placement of an arterial line in order to obtain the arterial blood pressure waveform. This invasive procedure is time-consuming, and presents a risk of infection to the patient. Respiratory variation in a photoplethysmograph (PPG or “pleth”) signal may provide a non-invasive alternative to PPV. The PPG signal can be obtained non-invasively, such as from a pulse oximeter. Respiratory variations of the PPG signal may be identified and measured in order to calculate one or more pleth-derived fluid responsiveness metrics.

However, these metrics may vary in scale as compared to the more well-known PPV metric. As a result, a pleth-based fluid responsiveness metric may provide a different numerical threshold for fluid administration as compared to a different pleth-based metric, or PPV. This variation can cause confusion in a clinical setting. Accordingly, there is a need for a reliable pleth-based fluid responsiveness metric that correlates well with PPV, which can be used to predict a patient's hemodynamic response to volume expansion, prior to fluid therapy.

### SUMMARY

The present invention relates to physiological signal processing, and in particular to methods and systems for processing physiological signals to predict a fluid responsiveness of a patient. In an embodiment, a medical monitoring system receives a photoplethysmography (PPG) signal, representing light attenuated by the patient's tissue, and analyzes respiratory variations in the PPG signal in order to predict a fluid responsiveness of the patient. The system calculates a fluid responsiveness predictor (FRP) value, and optionally displays this value to a clinician for use in determining the patient's likely response to fluid therapy. The system also determines a relationship between the FRP and pulse pressure variation (PPV), and adjusts the calculation of the FRP in order to map or scale an FRP threshold to a PPV threshold. This mapping provides an FRP metric with strong correlation to PPV, for non-invasive prediction of a patient's likely response to fluid loading.

In an embodiment, a method for predicting a fluid responsiveness of a patient includes receiving a photoplethysmograph (PPG) signal representing light absorption by a patient's tissue, identifying from the PPG signal a maximum heart rate and a minimum heart rate during a respiratory cycle, calculating a respiratory sinus arrhythmia metric based on the maximum and minimum heart rates, and displaying the metric as an indicator of a patient's likely fluid responsiveness.

In an embodiment, a method for predicting a fluid responsiveness of a patient includes receiving a photoplethysmograph (PPG) signal representing light absorption by a patient's tissue, identifying from the PPG signal a maximum and a minimum slope transit time during a respiratory cycle, calculating an FRP metric based on the maximum and minimum slope transit times, and displaying the FRP metric as an indicator of a patient's likely fluid responsiveness.

In an embodiment, a medical monitor for monitoring a patient includes an input receiving a photoplethysmograph (PPG) signal representing light absorption by a patient's tissue, and a fluid responsiveness predictor (FRP) calculator programmed to calculate an FRP metric. The monitor also includes a memory storing a relationship between the FRP metric and a pulse pressure variation (PPV) metric. The FRP metric is calculated based on a respiratory variation of the PPG signal and based on the relationship.

In an embodiment, a method for predicting a fluid responsiveness of a patient includes receiving a photoplethysmograph (PPG) signal responsive to light absorption by a patient's tissue, and identifying a respiratory-induced variation of the PPG signal. The method also includes storing a relationship between a fluid responsiveness metric and a pulse pressure variation, and determining a value of the fluid responsiveness metric based on the respiratory-induced variation and the relationship.

In an embodiment, a medical monitor for monitoring vital signs of a patient includes an electrical input providing a photoplethysmography (PPG) signal responsive to light absorption by a patient's tissue, a fluid responsiveness calculator programmed to calculate a Delta POP (DPOP) value based on a respiratory variation of the PPG signal, a scaling unit operating on the DPOP value to provide a scaled DPOP based on a relationship between DPOP and pulse pressure variation, and an output for providing the DPOP value or the scaled DPOP value to a display.

### BRIEF DESCRIPTION OF THE DRAWINGS

FIG. 1 illustrates a representation of a PPG signal, according to an embodiment of the present disclosure.

FIG. 2 illustrates a chart of a fluid responsiveness predictor versus pulse pressure variation, according to an embodiment of the present disclosure.

FIG. 3A illustrates a chart of a first fluid responsiveness predictor versus pulse pressure variation, according to an embodiment of the present disclosure.

FIG. 3B illustrates a chart of a second fluid responsiveness predictor versus pulse pressure variation, according to an embodiment of the present disclosure.

FIG. 3C illustrates a chart of a third fluid responsiveness predictor versus pulse pressure variation, according to an embodiment of the present disclosure.

FIG. 4 illustrates a chart showing an adjusted fluid responsiveness predictor, according to an embodiment of the present disclosure.

FIG. 5 illustrates a chart showing an adjusted fluid responsiveness predictor, according to an embodiment of the present disclosure.

FIG. 6 illustrates a flowchart showing a method for determining a relationship between an FRP and PPV, according to an embodiment of the present disclosure.

FIG. 7 illustrates a flowchart showing a method for determining a scaled fluid responsiveness predictor, according to an embodiment of the present disclosure.

FIG. 8 illustrates a flowchart showing a method for choosing settings for calculating a fluid responsiveness predictor, according to an embodiment of the present disclosure.

FIG. 9 illustrates a flowchart showing a method for determining a scaled fluid responsiveness predictor, according to an embodiment of the present disclosure.

FIG. 10 illustrates a block diagram of a system for determining a scaled fluid responsiveness predictor, according to an embodiment of the present disclosure.

FIG. 11 illustrates attributes of a cardiac pulse, according to an embodiment of the present disclosure.

FIG. 12 illustrates a plot of slope transit time (STT) over time, according to an embodiment of the present disclosure.

FIG. 13 illustrates a plot of slope transit time variation (STTV) versus pulse pressure variation, according to an embodiment of the present disclosure.

FIG. 14 illustrates an area calculation based on a derivative of a plethysmograph signal, according to an embodiment of the present disclosure.

FIG. 15 illustrates a plot of respiratory sinus arrhythmia (RSA) versus pulse pressure variation, according to an embodiment of the present disclosure.

FIG. 16 illustrates an isometric view of a pulse oximetry system, according to an embodiment of the present disclosure.

FIG. 17 illustrates a block diagram of a pulse oximetry system, according to an embodiment of the present disclosure.

### DETAILED DESCRIPTION

The present invention relates to physiological signal processing, and in particular to methods and systems for processing physiological signals to predict a fluid responsiveness of a patient. In an embodiment, a medical monitoring system receives a photoplethysmography (PPG) signal, representing light attenuated by the patient's tissue, and analyzes respiratory variations in the PPG signal in order to predict a fluid responsiveness of the patient. The system calculates a fluid responsiveness predictor (FRP) value, and optionally displays this value to a clinician for use in determining the patient's likely response to fluid therapy. The system also determines a relationship between the FRP and pulse pressure variation (PPV), and adjusts the calculation of the FRP in order to map or scale an FRP threshold to a PPV threshold. This mapping provides an FRP metric with strong correlation to PPV, for non-invasive prediction of a patient's likely response to fluid loading.

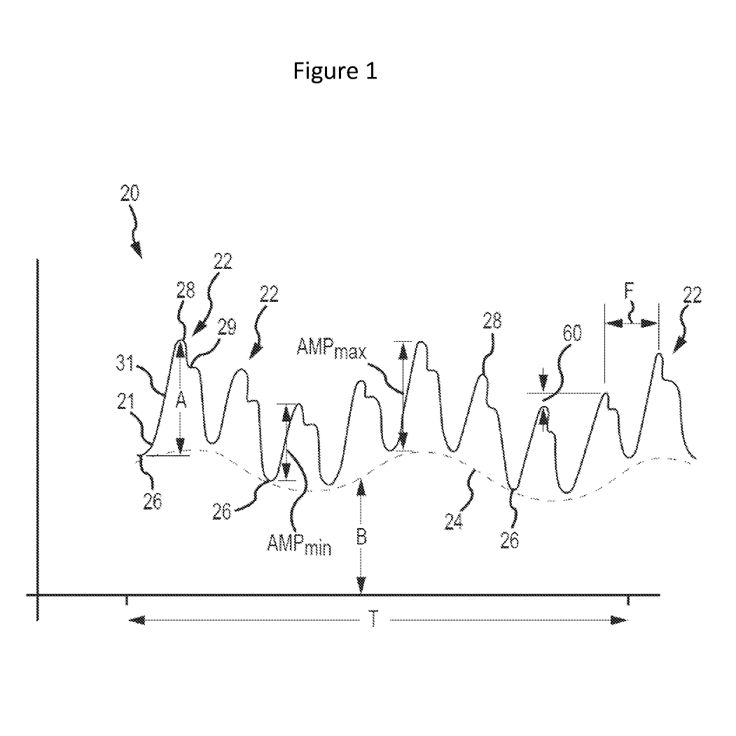

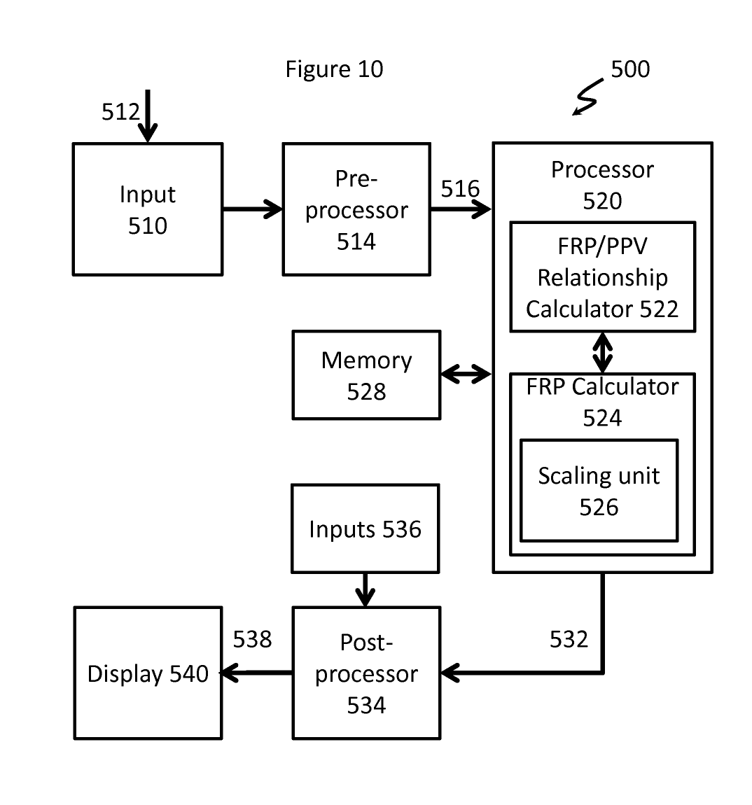

The photoplethysmography (PPG) signal can be obtained non-invasively by detecting light emitted into and emerging from a patient's tissue. An example of a device that can obtain a PPG signal is a pulse oximeter. Another example is a volume clamping device used to estimate blood pressure or cardiac output such as the Nexfin device (BMEYE, Amsterdam, Netherlands). An example of a PPG signal 20 obtained from a pulse oximeter is shown in FIG. 1. The PPG signal 20 may be output or represented as a PPG waveform 21 which represents the absorption of light by a patient's tissue over time. The PPG waveform 21 includes cardiac pulses 22, where absorption of light increases due to the increased volume of blood in the arterial blood vessel due to the cardiac pulse 22. Each cardiac pulse 22 may be identified based on a valley 26, peak 28, dicrotic notch 29, and subsequent valley 26. The PPG signal includes an upstroke 31 with an amplitude A, measured from the preceding valley 26 to the peak 28. Other amplitude values may be derived from the PPG waveform, such as downstroke amplitude, average amplitude, or area under the pulse 22. The PPG waveform 21 also includes a baseline shift B indicating a baseline level 24 of the light absorption. The PPG waveform 21 modulates above the baseline 24 due to the arterial blood pulses. The PPG waveform 21 shown in FIG. 1 may be the PPG signal 512 of FIG. 10 (discussed below). In other embodiments, other types of signals other than a PPG signal may be used as the input signal 512, such as a capacitance signal reflective of cardiac pulses from the subject. Where a PPG signal is discussed herein, it should be understood that another type of cardiac signal, in particular a non-invasive cardiac signal, may be used.

For some patients, the PPG signal 20 is affected by the patient's respiration, such as by inhaling and exhaling. A segment of a PPG waveform 21 during normal breathing is shown in FIG. 1. The waveform 21 includes the cardiac pulses 22. It should be noted that the number of cardiac pulses 22 per breath is not necessarily to scale, and may vary from patient to patient. Respiration may cause modulations in the PPG waveform 21.

One respiratory modulation is a modulation of the baseline B of the PPG waveform 21. The effect of the patient's breathing in and out causes the baseline 24 of the waveform 21 to move up and down, cyclically, with the patient's respiration rate. The baseline 24 may be tracked by following any component of the PPG waveform 21, such as the peaks 28, valleys 26, dicrotic notches 29, median value, or other value. A second respiration-induced modulation of the PPG signal 20 is a modulation of the amplitude A. As the patient breathes in and out, the amplitude A of the upstroke of each cardiac pulse 22 decreases and increases, with larger amplitudes tending to coincide with the top of the baseline shift B, and smaller amplitudes tending to coincide with the bottom of the baseline shift B (though the larger and smaller amplitudes do not necessarily fall at the top and bottom of the baseline shift). A third respiratory modulation is modulation of the frequency F between cardiac pulses, with cardiac pulses tending to have a higher frequency (shorter duration between pulses) during inhalation and a lower frequency (longer duration between pulses) during exhalation. Each of these modulations may be referred to as a respiratory component of the PPG signal 20, or a respiratory-induced modulation of the PPG signal 20. It should be noted that a particular individual may exhibit only the baseline modulation, or only the amplitude modulation, or only the frequency modulation, or combinations of these. As referred to herein, a respiratory component of the PPG signal 20 includes any one of these respiratory-induced modulations of the PPG waveform 21, a measure of these modulations, or a combination of them, such as an average or weighted average.

The respiratory modulations of the PPG waveform 21 can be affected by a patient's fluid responsiveness. For example, a patient that is fluid responsive (for example, a hypovolemic patient) may exhibit relatively larger respiratory variations of the PPG waveform 21, while a patient that is not fluid responsive may exhibit relatively smaller respiratory variations of the PPG waveform 21. When a patient loses fluid, the respiratory variations present in the patient's PPG signal 20 tend to increase. As an example, when the patient's fluid volume is low, the arterial system exhibits larger compliance and thus expands more with each cardiac pulse, relative to the baseline 24. Both the baseline modulation and the amplitude modulation may become more pronounced when a patient's fluid volume decreases. Thus, larger respiratory modulations may indicate that a patient is in need of fluids, while smaller respiratory modulations may indicate that a patient is not in need of fluids. The respiratory modulations of the PPG signal 20, such as the PPG waveform 21, may be identified and used to predict a patient's fluid responsiveness.

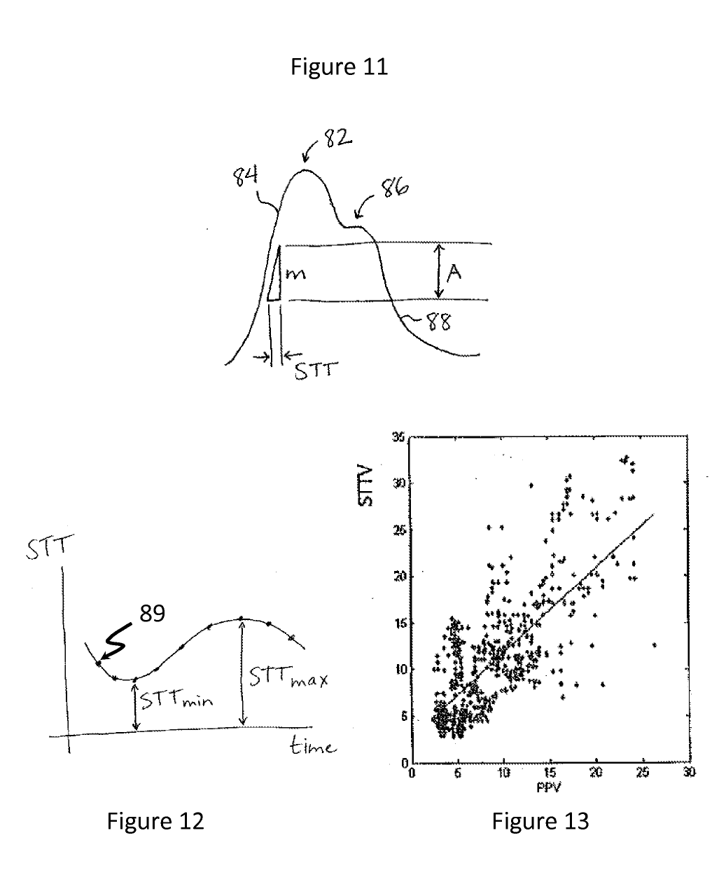

In an embodiment, a medical monitoring system receives a PPG signal and calculates a fluid responsiveness predictor (FRP) based on the PPG signal. In an embodiment, the FRP is a measure of a patient's likelihood of response to fluid therapy. As an example, the FRP represents a prediction of whether such fluid therapy will improve blood flow within the patient. In an embodiment, the FRP is a metric that reflects a degree of respiratory variation of the PPG signal, or a non-invasive cardiac signal. One example of an FRP metric is a measure of the amplitude modulations of the PPG signal, such as Delta POP (DPOP or APOP, defined below). In other embodiments, the FRP metric is a measure of the respiratory variation of the PPG, such as a measure of the baseline modulation of the PPG, a measure of slope transit time variation (described below with reference to FIGS. 11-14), respiratory sinus arrhythmia variation (described below with reference to FIG. 15), or suitable metrics assessing the respiratory modulation of the PPG. For example, an FRP may be based on the amplitudes or areas of acceptable cardiac pulses 22 within a particular time frame or window. The minimum amplitude of the cardiac pulses 22 may be subtracted from the maximum amplitude then divided by an average or mean value. Alternatively, an FRP may be derived from a frequency of cardiac pulses 22 within a time frame or window. For example, a modulation or variation in frequency among two or more cardiac pulses 22 may be used to derive an FRP. In general, the FRP may be based on one or more respiratory variations exhibited by the PPG signal 20. Further, an FRP may be determined through the use of wavelet transforms, such as described in United States Patent Application Publication No. 2010/0324827, entitled “Fluid Responsiveness Measure,” which is hereby incorporated by reference in its entirety.

In an embodiment, DPOP is used as the FRP. The DPOP metric is calculated from the PPG waveform 21 for a particular time window as follows:

DPOP=(AMPₘₐₓ−AMPₘᵢₙ)/AMPₐᵥₑ (1)

where AMPₘₐₓ represents the maximum upstroke amplitude (amplitude from a pulse minimum to a pulse maximum) during the time window (such as time window T in FIG. 1), AMPₘᵢₙ represents the minimum upstroke pulse amplitude during the time window, and AMPₐᵥₑ is the average of the two, as follows:

AMPₐᵥₑ=(AMPₘₐₓ+AMPₘᵢₙ)/2 (2)

In other embodiments, AMPₘₐₓ and AMPₘᵢₙ may be measured at other locations of the PPG, such as within or along a pulse. DPOP is a measure of the respiratory variation in the AC portion of the PPG signal. DPOP is a unit-less value, and can be expressed as a percentage. In an embodiment, the time window is one respiratory cycle (inhalation and exhalation). In an embodiment, the time window is a fixed duration of time that approximates one respiratory cycle, such as 5 seconds, 10 seconds, or another duration. In other embodiments, the time window may be adjusted dynamically based on the patient's calculated or measured respiration rate, so that the time window is approximately the same as one respiratory cycle. A signal turning point detector may be used to identify the maximum and minimum points in the PPG signal, in order to calculate the upstroke amplitudes. In some embodiments, AMPₘₐₓ and AMPₘᵢₙ may be calculated by identifying a maximum value and a minimum value within a cardiac pulse window, and calculating a difference between those values. This difference may correspond with an upstroke or a downstroke, for example.

To assess the usefulness of a fluid responsiveness predictor, such as DPOP, the FRP can be compared with PPV (pulse pressure variation), a metric that is obtained from the invasive arterial pressure waveform and that is known to reliably indicate a patient's fluid responsiveness. DPOP and PPV have the same mathematical formulation, but are taken from different signals (DPOP from the PPG signal, and PPV from the invasive arterial pressure signal).

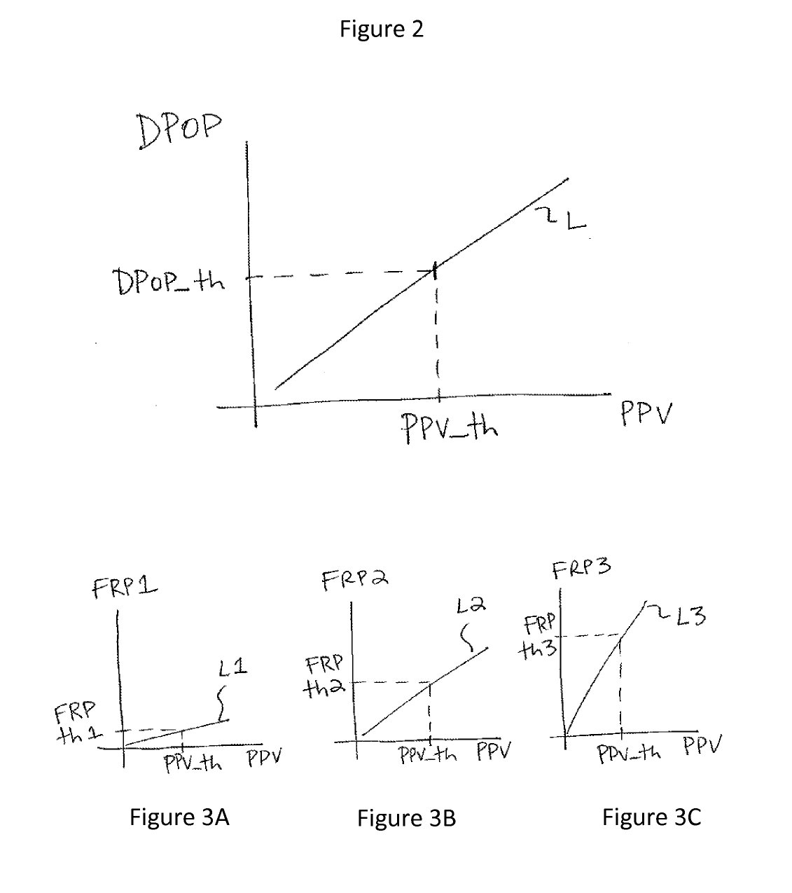

FIG. 2 shows a plot of DPOP versus PPV, to illustrate the correlation between the two metrics. The relationship between the two metrics is indicated by the best fit line L. This best fit line is based on data points from historical patient data. The trend line L shows a strong correlation between PPV and DPOP. FIG. 2 also shows an indication of a threshold value of PPV, labeled PPV_th, and a corresponding threshold value of DPOP, labeled DPOP_th. The PPV threshold value PPV_th is a recognized threshold value that indicates whether the patient is likely to be fluid responsive or not. Based on the correlation between PPV and DPOP, the corresponding DPOP threshold value DPOP_th can also be used as a threshold indicator of fluid responsiveness. For example, if the calculated or displayed DPOP (or PPV) value is greater than the threshold DPOP_th (or PPV_th), then the patient is likely to benefit from fluid therapy. If the displayed DPOP (or PPV) value is less than DPOP_th (or PPV_th), the patient may not benefit. Based on this determination, fluid administration may be initiated, continued, or ceased.

The DPOP_th number may not be the same as the PPV_th number. For example, PPV_th may be 13%, while the DPOP_th is 15%. When this is the case, caregivers such as nurses, doctors, and clinicians must remember the two different values and take appropriate action when the respective threshold is crossed. This is a potential point of confusion, as caregivers who are familiar with PPV and new to the use of DPOP may be inclined to provide fluids when the DPOP value crosses PPV_th value, instead of the DPOP_th value.

Furthermore, DPOP is only one example of a PPG-based FRP, and other FRP values, or even differing formulations of DPOP, may exhibit differing relationships with PPV, leading to different threshold values. This is illustrated in FIGS. 3A-3C. FIG. 3A shows a plot of a first FRP metric, FRP1, versus PPV, and a first threshold value FRP_th1 that correlates with the established PPV threshold value PPV_th. FIG. 3B shows a plot of a second FRP metric, FRP2, versus PPV, and a second threshold value FRP_th2 that correlates with PPV_th. FIG. 3C shows a plot of a third FRP metric, FRP3, versus PPV, and a third threshold value FRP_th3 that correlates with PPV_th. FRP_th3 is greater than FRP_th2, which is greater than FRP_th1, even though each of these thresholds corresponds to the PPV_th value.

The variation in threshold values FRP_th1, FRP_th2, and FRP_th3 is due to the different relationships between different FRP metrics and PPV, as shown by the three different fit lines L1, L2, and L3. Because the relationship with PPV may vary with different FRP metrics, the FRP threshold value is not necessarily consistent across these different FRP metrics. As a result, an FRP metric based on a first respiratory variation in the PPG signal may have a different threshold value (for example, 10% than an FRP metric based on a second, different respiratory variation in the PPG signal (with a threshold of, for example, 20%). While these different FRP values may all correlate with PPV, and thus provide a reliable indication of fluid responsiveness, it may be difficult for a caregiver to remember which threshold value is applicable at any given time.

According to an embodiment of the present disclosure, a method is provided for scaling an FRP metric to a defined relationship with PPV. Various FRP metrics can each be scaled appropriately to bring them all to the same scaled correlation with PPV, such that the FRP metrics all exhibit the same threshold value. As a result, caregivers do not need to re-calibrate their practices based on the particular FRP being used. Additionally, other factors that affect the correlation between FRP and PPV, such as patient characteristics or PPG sensor type, can also be taken into account to scale the calculated FRP value back to the same, consistent threshold.

FIG. 5 illustrates a plot of FRP and an adjusted FRP versus PPV, according to an embodiment of the present disclosure. Raw data points 52 are represented in FIG. 5, based on a calculation of a respiratory-induced variation in the PPG signal—in this case, DPOP. In FIG. 5, a best fit line L6 with slope m is plotted through the raw data points 52, crossing the y-axis at value c. This line L6 can be identified with a least squares regression or a least-median-of-squares regression. This raw data exhibits a threshold value FRP_th_{raw} that correlates with the PPV threshold PPV_th of 13%. However, the illustrated FRP_th_{raw} value is much greater than 13%.

Accordingly, the raw data can be re-scaled to adjusted data points 52′ having an adjusted fit line L7 that passes through the origin with a slope n. In an embodiment, n=1 or close to 1. In the example of FIG. 5, this modification is accomplished by shifting and re-scaling the raw FRP data, as follows:

FRP_{adjusted}=(FRP_{raw}−c)*n/m (3)

where m and n are the slopes of lines L6 and L7, respectively, and c is the FRP value where line L6 crosses the y-axis. This adjustment shifts the raw data down by an amount c and then re-scales it according to the slopes n and m. For the particular data set plotted in FIG. 5, m=1.07, c=8.568, and n=1.0.

As another example, the raw FRP values may simply be shifted up or down by the difference between the raw FRP threshold and the PPV threshold, as follows:

FRP_{adjusted}=FRP_{raw}+(PPV_th−FRP_th_{raw}) (4)

The method outlined in FIG. 5 enables the raw FRP data to be scaled and/or shifted to better match the FRP and PPV thresholds. However, the best fit line L6 through the raw data 52 is likely not the best representation of the FRP-PPV relationship, as it does not pass through the origin. When the PPV value is zero (indicating zero respiratory modulations in the blood pressure waveform), the FRP value should also be zero (indicating zero respiratory modulations in the PPG waveform). The line L6 is likely inflated upward above the origin due to noise in the data, which may be non-symmetric tending to positive errors in DPOP for a given PPV. The true relationship for the data points 52 likely exists somewhere nearer a lower bound.

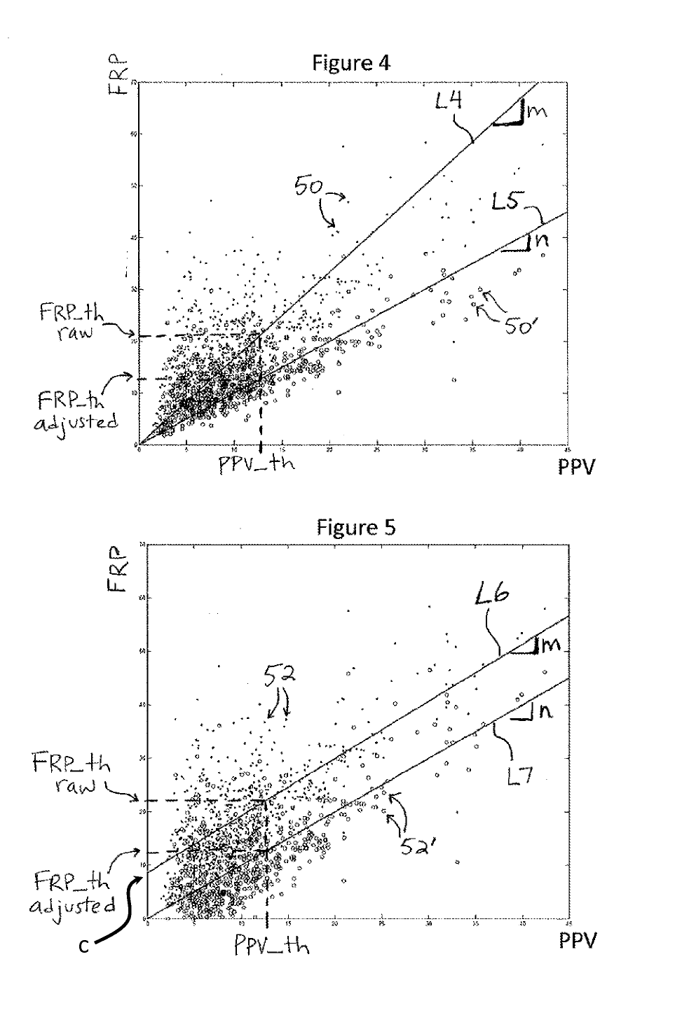

FIG. 4 illustrates a plot of FRP and an adjusted FRP versus PPV, with a line fit L4 passing through the origin, according to an embodiment of the present disclosure. Raw data points 50 are represented in FIG. 4, based on a calculation of a respiratory-induced variation in the PPG signal, in this case DPOP. In FIG. 4, the best fit line L4 with slope m is plotted passing through the origin and the raw FRP data points 50. This line L4 can be identified with a least squares regression forcing the abscissa crossing through the origin. The line L4 can also be identified with a least-median-of-squares regression, also forced through the origin, to counteract the disproportionate effect of outliers on the data.

The fit line L4 through the raw data 50 in FIG. 4 exhibits a threshold value FRP_th_{raw} that correlates with the PPV threshold PPV_th of 13%. However, this FRP_th_{raw} value is much higher than 13%. Accordingly, the raw data can be re-scaled to adjusted data points 50′ with an adjusted fit line L5 that passes through the origin with a slope n of 1 or close to 1. By re-scaling the raw data to this fit line L5, the raw threshold value FRP_th_{raw} is scaled to an adjusted value FRP_th_{adjusted} that corresponds to the PPV threshold value PPV_th. This scaled FRP data can be presented to the caregiver so that decisions about fluid therapy can be made based on the same threshold value that caregivers are familiar with for PPV. Thus, the FRP value can be modified to mimic the PPV value. In the example of FIG. 4, when the slope n=1 (or close to 1), this modification is accomplished by re-scaling the raw FRP data according to the slope or gradient, m, of the original raw data, as follows:

FRP_{modified}=FRP_{raw}/m (5)

If the slope n of the line L5 is 1 or close to 1, the FRP_th_{adjusted} can be adjusted to equal or match, or closely match, the value of PPV_th. If a different value of the FRP_th is desired, the slope n can be different than 1. In such an instance, the modified FRP values are calculated as follows:

FRP_{modified}=FRP_{raw}*n/m (6)

For the particular data plotted in FIG. 4, m=1.667 and n=1.0.

In an embodiment, when other FRP metrics are then mapped or scaled, the same slope value n is used such that the various FRP metrics each map to the same threshold value FRP_th_{modified}. This modified FRP threshold value need not be exactly the same value as the PPV threshold value, but it is helpful to caregivers if the FRP threshold value is kept consistent across differing FRP formulations. For example, the PPV threshold PPV_th may be 13%, while the FRP threshold FRP_th_{modified} is 15%, as long as the modified FRP threshold value is kept the same or consistent, not overly varying. In another embodiment, the FRP threshold FRP_th_{modified} is adjusted to 13% to match the PPV threshold.

In an embodiment, when the threshold value of the raw FRP data is known, the adjustment can be accomplished as follows:

FRP_{modified}=FRP_{raw}*(PPV_th/FRP_th_{raw}) (7)

As shown in FIGS. 4 and 5, and Equations 3-7, the modification or adjustment to the raw FRP values may move them up or down, shift them, scale them, rotate them, or any combination of these adjustments, in order to bring the FRP threshold value into a desired relationship with the PPV threshold.

The data points 50, 52 and fit lines L4, L6 in FIGS. 4 and 5 may be derived from historical patient data, such as historical databases that include PPV and FRP values for a patient population. Similarly, the independent variables in Equations 3-7 may be derived from historical patient data. Alternatively, or in addition, these values, fit lines, and/or data points may be collected from an individual patient over time, to produce a scaled relationship tailored to that individual patient. Once these various inputs or combinations of inputs have been used to identify a relationship between FRP and PPV, that relationship can be stored and used in future monitoring sessions to calculate a modified or adjusted FRP value with a consistent threshold value for predicting a patient's fluid responsiveness.

The examples given above utilize raw FRP values to identify a relationship between FRP and PPV, and then formulate a mapping relationship to map the raw FRP values to mimic PPV. This mapping relationship may include shifting the raw data, scaling the raw data, rotating the raw data, or combinations of these operations. Once this relationship is identified, new FRP values may be calculated by first calculating a raw FRP value and then adjusting that raw value with the identified relationship. It is also an option for the identified relationship to be programmed into the original raw FRP calculation. For example, using DPOP as an example FRP, new values of DPOP may be calculated by adding a scaling or shifting factor to Equation 1, rather than first calculating a raw DPOP and then separately scaling it in two steps. Either approach is acceptable.

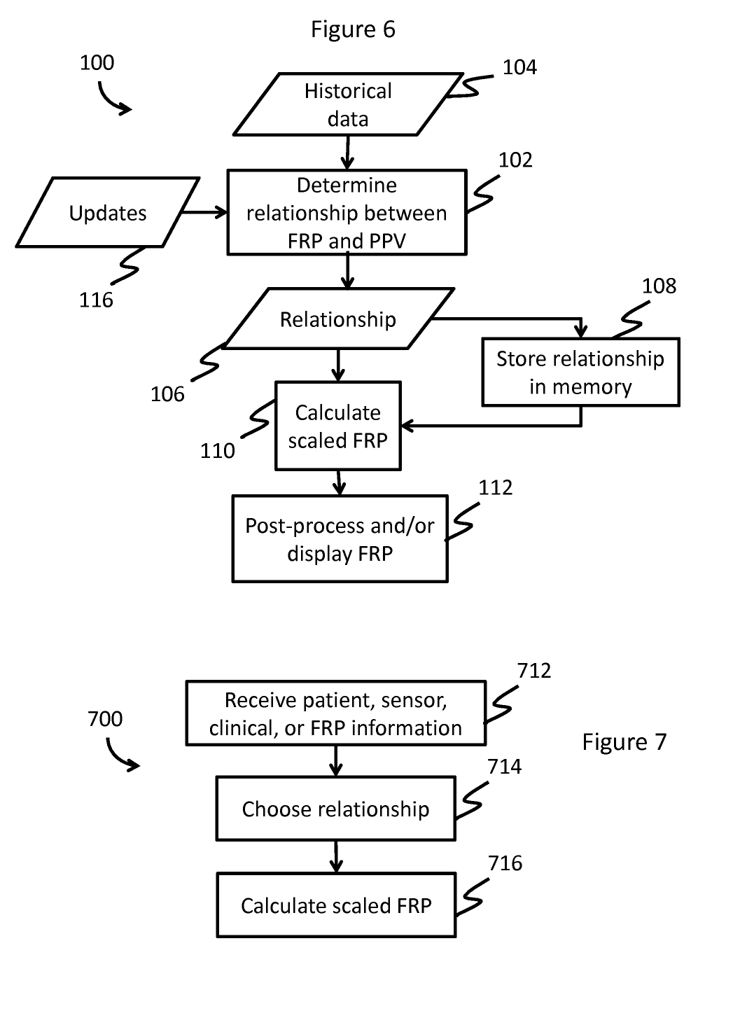

A method 100 for determining an FRP value is illustrated in FIG. 6, according to an embodiment. The method includes determining a relationship between the FRP and PPV, at 102. Historical patient data 104 may be taken as an input for this process. Examples of determining a relationship are discussed above. The relationship 106 is output and may be stored in memory at 108, such as by storing a scaling factor, a modified equation, a series of equations, or a table. This identified relationship is used to calculate a scaled FRP at 110. The scaled FRP value may be post-processed (discussed in more detail below), and/or displayed or output for display at 112.

In an embodiment, the identified relationship between the FRP and PPV is used to map differing FRP values to the same, consistent relationship with PPV, in order to provide a consistent FRP threshold value. This ability to scale, shift, or map an FRP value to a defined relationship can also be useful for updating the process with new or different steps. For example, updates 116 are shown in FIG. 6. These updates may, for example, include a different formulation of the FRP metric, new data from a new patient group, software updates, calibration updates, or other changes. Such an update or change could lead to a shift in the clinically-relevant FRP threshold value. For example, the new patient group may exhibit fluid responsiveness above a different value, such as 18%, compared to an accepted or standard value, such as 15%. In an embodiment, the method includes inputting these updates 116, and repeating the process of determining a relationship at 102. If the updates 116 affect the relationship, a new relationship is identified, stored, and used to calculate FRP values that scale to the previous threshold value. As a result, updates can be provided without shifting the clinical FRP threshold. Caregivers can continue to rely on the threshold value (such as 13% or 15% or 17% or any suitable value) with which they are familiar.

Various relationships can be identified and stored and then selected for use based on the patient being monitored, the conditions of monitoring, and/or the particular FRP metric being used. A method 700 for calculating a scaled FRP according to an embodiment is shown in FIG. 7. The method includes receiving patient, sensor, clinical, and/or FRP information at 712, choosing a corresponding stored relationship at 714, and calculating a scaled FRP based on that relationship at 716. The information received at 712 may be automatically detected (such as information stored on a sensor), pre-assigned (such as information assigned to a monitor that is located in an operating room), or user-inputted.

Patient information may include relevant physiologic information that may affect the FRP calculation, such as skin pigmentation, patient temperature, patient heart arrhythmia, circulatory compromise or disease, prescribed vasoactive drugs, circulatory support (LVAD (left ventricular assist device), IABP (intra-aortic balloon pump)) or other patient information. Based on analysis of historical patient data, these factors may influence the scaling between the FRP and PPV. Clinical information may include patient position, room temperature, the location within the hospital, such as an operating room or a general care floor, or environmental conditions.

FRP information refers to different bases for calculating an FRP, such as DPOP, STTV (described below), RSA (described below), or metrics assessing respiratory modulations of a cardiac signal. Based on the FRP type, an adjustment to the scaling factor may be needed, to align the FRP threshold with the PPV threshold. The FRP type may be automatically selected, may be pre-assigned, or may be selected by a user.

Sensor information may include sensor type, such as sensors tailored for particular locations on the patient's body (for example, fingers, toes, forehead, or ear), or for certain patient groups (neonates, children, adults). The resulting PPG signals from these various different sensors may exhibit different properties, and as a result, the FRP calculation based on these different PPG signals may be adjusted.

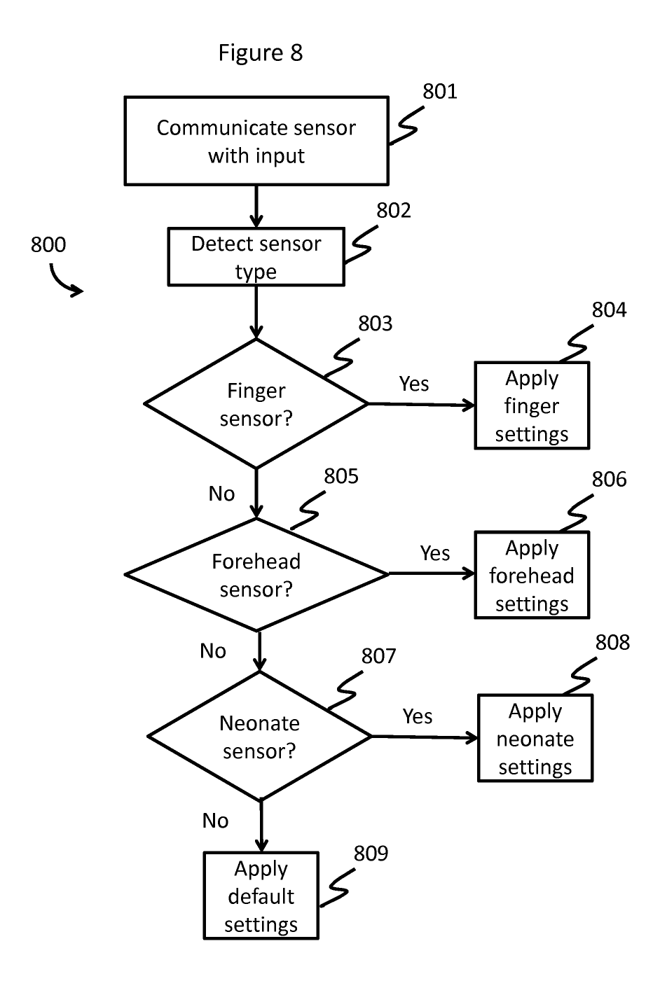

An example of an adjustment based on sensor type is outlined in FIG. 8. It should be understood that adjustment of the FRP settings may also take place for patient information, clinical information, and FRP type, as mentioned above. In FIG. 8, the method 800 includes communicating a sensor with a sensor input, at 801. The method then includes detecting a sensor type, at 802. As noted above, examples of sensor type include forehead, finger, toe, ear, nose, neonate, pediatric, and adult sensors. Information about the sensor type may be stored on the sensor itself (such as a lookup table with coefficients, stored on a memory chip on the sensor or sensor cable), and communicated to the processor when the sensor is connected to the input. That is, the sensor may identify itself as a particular type, and provide the associated settings to the processor for operating the FRP calculation. Alternatively, the processor may determine the sensor type and retrieve the associated settings. Alternatively, the user may specify the type of sensor by inputting that information.

The method then includes choosing the settings associated with the identified type. A few examples are outlined in FIG. 8. For example, the method may include applying finger settings, at 804, if a finger sensor is identified at 803. As another example, the method may include applying forehead settings, at 806, if a forehead sensor is identified at 805. As another example, the method may include applying neonate settings, at 808, if a neonate sensor is identified at 807. Other sensor types, environmental settings, patient information, and FRP types may be included, though they are not all outlined in FIG. 8. If no sensor type or other relevant information is identified, the method may include applying default settings, at 809. Default settings may be those for an adult finger sensor, on a general care floor, calculating DPOP as the FRP, for example.

The settings that are applied for a particular sensor type are settings that adjust the FRP calculation to accommodate differences in the PPG signal from that particular type of sensor. For example, the settings may include a different scaling factor for bringing the FRP threshold into alignment with the PPV threshold. This scaling factor for a forehead sensor may differ from the scaling factor for a finger sensor. Other settings that may be adjusted include the amount or type of pre-filtering, such as the reducing the amount of low-pass filtering on the PPG signal before processing it for FRP calculations.

As another example, when a non-finger sensor is detected (such as a forehead or ear sensor), differences in the resulting PPG signal may scale the FRP calculation, as compared to a finger sensor. The PPG signal from a forehead sensor may exhibit smaller peak-to-peak amplitude, or different respiratory modulations, than a finger sensor, resulting in a different DPOP number, for example. The PPG signal from an ear sensor may exhibit peaks with a more rounded shape, and different amplitudes, than a finger sensor. As a result, the settings for these sensors may include applying a different scaling factor. A scaling factor can be chosen for each particular sensor type, based on historical patient data, or patient-specific data if available, showing the relationship between DPOP values calculated from a finger sensor and from the particular non-finger sensor. Thus, DPOP values from various sensor types can be mapped to the same PPV scale.

As another example, when a neonatal sensor is detected, the settings may be adjusted to provide a different time window for the FRP calculation. As described above, an FRP may be based on the amplitudes or areas of acceptable cardiac pulses within a particular time frame or window. Neonates tend to have a higher pulse rate than adults, and thus this time window may be decreased when a neonate sensor is detected, to reduce the number of cardiac pulses present in the window. Similarly, when a pediatric sensor is detected, the window may also be decreased, to a lesser extent than a for neonate sensor.

Accordingly, an FRP system according to an embodiment includes alternate code modules associated with alternate sensor types, patient groups, and other relevant factors. The code modules include different, additional, or fewer steps for the calculation of the FRP parameter, according to the clinical environment, sensor type, and patient group. A method of calculating an FRP, according to an embodiment, includes adjusting settings (such as thresholds, coefficients, scaling factors, filtering, and other steps) according to a detected sensor type or other clinical or patient information. In an embodiment, the processor checks for the appropriate FRP settings prior to calculating and displaying an FRP value. If no FRP settings are detected on the sensor, then the processor may determine that the sensor is not an authorized or authentic sensor, and may display an appropriate warning message. This check prevents the use of sensors that are not properly calibrated for the FRP signal processing.

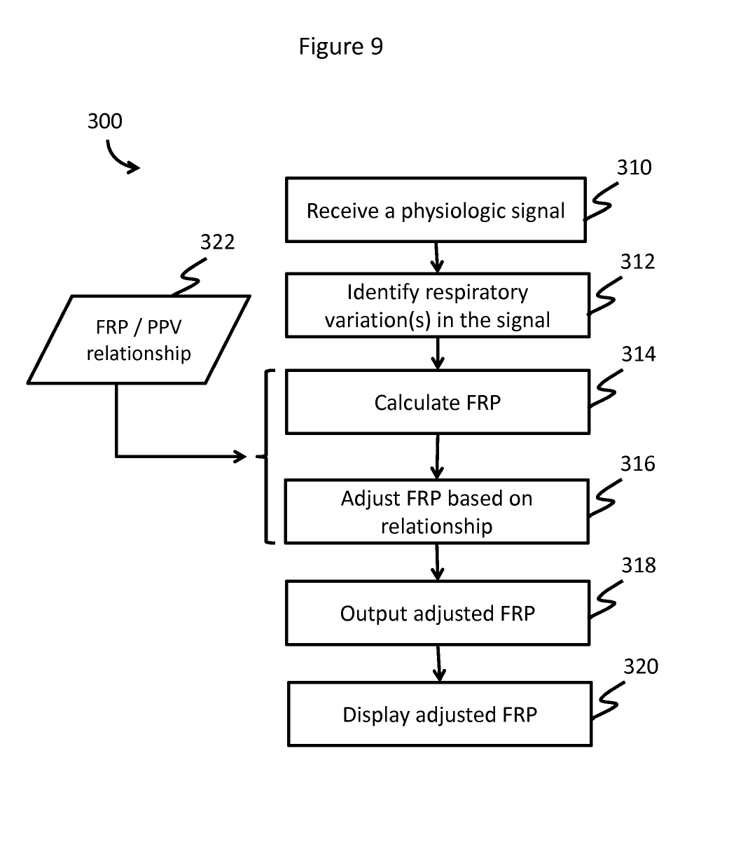

A method 300 for predicting a fluid responsiveness of a subject according to an embodiment is shown in FIG. 9. The method includes receiving a physiologic signal at 310, such as a PPG signal. Although not shown in the figure, the PPG signal may be pre-processed prior to further calculations, as described below with reference to FIG. 10. In FIG. 9, the method also includes identifying one or more respiratory variations in the physiologic signal at 312. Examples of respiratory variations of a PPG signal are described above and shown in FIG. 1. In FIG. 9, the method also includes calculating an FRP at 314, based on the identified respiratory variations. The method also includes adjusting or modifying the FRP at 316, based on a stored relationship 322. The stored relationship 322 may be a previously determined relationship between the FRP and PPV, based on the particular FRP being used, sensor type, patient information, or other inputs, as described in examples above. It should be noted that boxes 314 and 316 may be performed together in one step, rather than in separate steps. Optionally, the method also includes outputting the adjusted FRP at 318, and displaying the adjusted FRP at 320.

In an embodiment, the method of FIG. 9 is called at a specified frequency, such as the duration of a desired time window of PPG data. Over that window of data, the method operates to calculate the FRP and adjust it as necessary. In an embodiment, the method is called every 5 seconds and uses 10 seconds of data, thus incorporating both new and previous data into the data window. Other time windows may be used, and may be adjusted based on a patient's respiration rate.

A system 500 for monitoring a patient's vital signs, such as a patient's fluid responsiveness, according to an embodiment, is shown in FIG. 10. The system 500 includes an input 510 that receives a PPG signal 512. The sensor may be a pulse oximetry sensor applied to a patient's finger, toe, earlobe, or forehead, for example. The input may include a socket or port for wired connection to the sensor, a wireless receiver for receiving signals wirelessly from the sensor, or another suitable electrical input. The system also includes a pre-processor 514 that initially processes the PPG signal 512. For example, the pre-processor may include one or more filters, such as a low pass filter to remove noise, and/or a filter based on the patient's heart rate to remove irregular pulses. The pre-processor manipulates the incoming PPG signal prior to parameter calculations, such as heart rate, oxygen saturation, or FRP. Pre-processing may also include removing the dicrotic notches 29 (shown in FIG. 1), such as by removing smaller peaks within a defined proximity, such as 0.35 seconds, to a larger peak. The pre-processor may apply a mapping analysis to identify upstrokes in the PPG signal, such as by looking for peaks in the derivative of the PPG signal to identify separate upstrokes (a derivative fiducial detection method). Signal processing methods for identifying upstrokes and other methods are described in more detail in U.S. application Ser. No. 13/243,951 (U.S. Publication No. 2013/0080489).

The pre-processed PPG signal 516 is then passed to a parameter processor 520. In an embodiment, the processor 520 includes an FRP/PPV relationship calculator 522 an FRP calculator 524. The relationship calculator 522 may include a code module or engine programmed to identify a trend, such as a line fit, in historical data, and an operation for mapping that trend to a desired relationship with PPV. Examples of this mapping operation are described above. The relationship may be stored in a memory 528.

The FRP calculator 524 calculates an FRP value based on the PPG signal 516, as discussed above (and below). In an embodiment, the FRP calculator 524 includes a scaling unit 526, which applies a correction factor, adjustment, mapping operation, or modifier to the FRP value based on the relationship. In an embodiment, the scaling unit includes a table, or other storage mechanism, storing different FRP formulas, methods, or adjustments based on different FRP/PPV relationships. The applicable formula, method, or adjustment is selected and used to calculate the FRP, which is then provided through output 532. The output may be a transmission of the FRP value to another code module, another processor, memory, or display, for example. In an embodiment, the scaling unit 526 is incorporated into the post-processor 534 discussed below, rather than the parameter processor 520.

The system 500 may also include a post-processor 534 which further processes the FRP value to provide a smoothed or processed FRP value 538 prior to displaying it to a caregiver. This step can include filtering, smoothing, and/or averaging the FRP number, displaying the number, and/or displaying a trend. For example, the post-processor 534 may smooth the FRP value by calculating a running average of the calculated FRP values over a time window. The time window may be chosen by a user for a smoother or faster FRP value (for example, 120 seconds, or 15 seconds, or other similar durations). The post-processor may also remove outlier FRP values before averaging or displaying. For additional smoothing, the post-processor may employ percentile averaging, in which only the middle 50% of calculated FRP values within a time window are added to the running average, and the lowest 25% and highest 25% of values are removed.

Additionally, the post-processor may determine whether posting criteria are met, prior to posting the FRP value, in order to remove particular FRP values due to conditions that indicate a deterioration in the PPG signal or the patient's condition. If the posting criteria are not met, the new value of the FRP is discarded. In this case, if previous FRP values met the posting criteria, then the previously calculated FRP value may continue to be displayed. If new values continue to fail the posting criteria, a timer may be incremented until it reaches a threshold, such as 15, 20, 30, 45, or 60 seconds. At that time the previously displayed FRP value may be removed and no value displayed until new data that meets the posting criteria is received. Posting criteria include various checks to assess the likely accuracy of the newly calculated FRP number. Examples of posting criteria include an arrhythmia flag (indicating that cardiac arrhythmia may be present in the PPG signal), a signal-to-noise ratio value or artifact flag (indicating noise is present in the PPG signal), a servo flag (indicating that a recent gain change occurred within the current processing window, which could distort calculations based on the PPG amplitude), system flags (such as sensor off or sensor disconnected), and/or physiologic flags (such as heart rate, respiratory rate, or blood oxygen saturation being out of a specified range, above or below a threshold, zero, or undetected). These flags indicate that the FRP number may be distorted by signal degradation or a physiological event. The posting criteria may also include a cap for the FRP number itself; for example if the FRP number exceeds a threshold (such as 70%), then it is not posted. These various system, signal, and physiological inputs to the post-processor are labeled as inputs 536 in FIG. 9.

The system may also include an output that passes the processed FRP value 538 to a display 540 for displaying the FRP value to a caregiver, such as a doctor or nurse or other clinician, for making clinical decisions about patient care, as described above. The system 500 of FIG. 10 may provide a prompt on the display 540 when the FRP value crosses a defined FRP threshold. The FRP value may be used in GDT (goal-directed therapy) to incrementally load the patient until the FRP value indicates that further fluid therapy would not be helpful. The 15% threshold is merely an example, and it is to be understood that the threshold may be greater or less than 15%. Moreover, different thresholds may be used to determine whether individual patients would benefit from fluid administration. According to embodiments herein, a calculated FRP value is able to identify PPV values either side of a defined threshold with high sensitivity and specificity.

Referring again to FIG. 10, the block diagram illustrates modules that represent circuit modules that may be implemented as hardware and/or software. It should be noted that the various components of the system 500 may be connected via wired or wireless connections. The components may be separate from each other, or various combinations of components may be integrated together into a medical monitor or processor, or contained within a workstation with standard computer hardware (for example, processors, circuitry, logic circuits, memory, and the like). The system may include processing devices such as microprocessors, microcontrollers, integrated circuits, control units, memory (such as read-only and/or random access memory), and/or other hardware. One or more system components may be housed within a smart cable, a cable adapter, or the like, with a cable that connects to a sensor, such as a pulse oximetry sensor, at one end. Further, one or more system components may connect to an external device such as a cellular or smart phone, tablet, other handheld device, laptop computer, monitor, or the like that may be configured to receive data from the system and show the data on a display of the device.

The systems and methods described herein may be provided in the form of tangible and non-transitory machine-readable medium or media (such as a hard disk drive, etc.) having instructions recorded thereon for execution by a processor or computer. The set of instructions may include various commands that instruct the computer or processor as a processing machine to perform specific operations such as the methods and processes of the various embodiments of the subject matter described herein. The set of instructions may be in the form of a software program or application. The computer storage media may include volatile and non-volatile media, removable and non-removable media implemented in any method or technology for storage of information such as computer-readable instructions, data structures, program modules or other data. The computer storage media may include, but are not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which may be used to store desired information and that may be accessed by components of the system.

While some examples above discuss the correction factor applied to DPOP, other FRP metrics or combinations of metrics may be used and corrected accordingly. For example, amplitude values and/or modulations, or baseline values and/or modulations from the PPG signal, or various respiratory components of the PPG signal may be scaled according to their relationship with PPV, in order to provide an adjusted FRP metric. Also, two other FRP metrics, STTV and RSA, are discussed below.

The relationship between an FRP and PPV may be patient-specific, or may be predetermined, such as based on historical or clinical data. The relationship between may be represented by a non-linear polynomial function, or by a series of piecewise functions, or another type of mapping (non-parametric, non-linear, or heteroassociative), or the relationship may be learned by a neural network.

DPOP is discussed as an illustrative FRP in some examples above, but the FRP metric is not limited to DPOP. Another example of a non-invasive PPG-based FRP metric is slope transit time variation (STTV)—that is, a measure of the variation of slope transit time (STT), or the inverse of the gradient of the pulse upstroke. This metric is shown in FIG. 11. FIG. 11 shows a cardiac pulse 82 with an upstroke portion 84, dicrotic notch 86, and downstroke portion 88. A slope m can be identified per a single unit of amplitude A along the upstroke 84. STT is the inverse of this slope, per unit amplitude, calculated as STT=1/m. The slope m can be calculated from a received PPG signal by identifying the peak of the first derivative. Then STT is calculated to convert the slope into a time per unit amplitude. It should be noted that the PPG signal may be pre-filtered prior to the computation of STT (and STTV). In effect, STT measures a duration per unit amplitude change A, and indicates a transit time of the upstroke or slope of the PPG wave (hence the name “slope transit time”). STT increases when the upstroke of the pulse becomes less steep, and decreases when the upstroke of the pulse becomes more steep. STT is thus an indication of the shape of the cardiac pulse.

The shape of the cardiac pulse 82 in the PPG signal varies with respiration due to the interaction between blood pressure, heart rate, pulse transit time, and other factors. For some patients, on inhalation, blood pressure decreases, pulse transit time increases, and heart rate increases. The result of this interaction is that the slope m of the upstroke portion 84 decreases, causing an increase in STT. For some patients, on exhalation, blood pressure increases, pulse transit time decreases, and heart rate decreases. The result of this interaction is that the slope m of the upstroke portion 84 increases, causing a decrease in STT.

An example plot of STT values 89 calculated at each heart beat is shown in FIG. 12, according to an embodiment. FIG. 12 shows values of STT increase and decrease cyclically, with respiration. STT is a measure that reflects changes in the cardiac pulse corresponding to respiratory pressure changes. In the plot, the maximum STT value over one respiratory cycle is denoted STTₘₐₓ, and the minimum STT value over the same respiratory cycle is denoted STTₘᵢₙ. One respiratory cycle refers to a period of time encompassing a complete breath cycle, including one inhalation and one exhalation. STTₘₐₓ and STTₘᵢₙ may be identified over one respiratory cycle, or over another duration (fixed or adjustable) such as 5, 6, 7, 8, 9, 10 seconds, or other suitable durations. These values may be calculated over different respiratory cycles, such as two or more adjacent cycles. When the respiratory period is not accurately known, the duration over which STTₘₐₓ and STTₘᵢₙ are identified may be set as the longest physiologically likely breath, for example, 20 seconds.

The variation in STT is a respiratory-induced variation in the PPG signal and can be used as a predictor of fluid responsiveness. To quantify this variation, an STTV metric can be calculated as follows:

STTV=(STTₘₐₓ−STTₘᵢₙ)/STT_{avg}, where (8)

STT_{avg}=(STTₘₐₓ+STTₘᵢₙ)/2 (9)

A plot of STTV versus PPV is shown in FIG. 13, according to an embodiment. The plot was generated from data collected from a group of patients during the post-induction, pre-incision period in the operating room. The data points show a strong correlation between STTV and PPV, confirming that STTV is useful as an indicator of fluid responsiveness. In various embodiments, STTV may be used as the FRP identified herein. In particular, in FIG. 13, a threshold STTV value identifying likely fluid responsiveness may be identified, correlating to the PPV threshold value. This STTV threshold value may not match the same numerical threshold value of other FRP metrics. Accordingly, the STTV values may be scaled, shifted, rotated, or adjusted to map the STTV threshold value to a desired numerical value, such as the PPV threshold value, as described herein, in order to maintain a consistent threshold value indicating fluid responsiveness. In this way, whether a medical monitoring system utilizes DPOP or STTV (or another metric) as the FRP, a caregiver can expect to make clinical decisions based on the same numerical threshold, for example, 13% or 15%, rather than having to adjust clinical procedures based on the particular FRP being used.

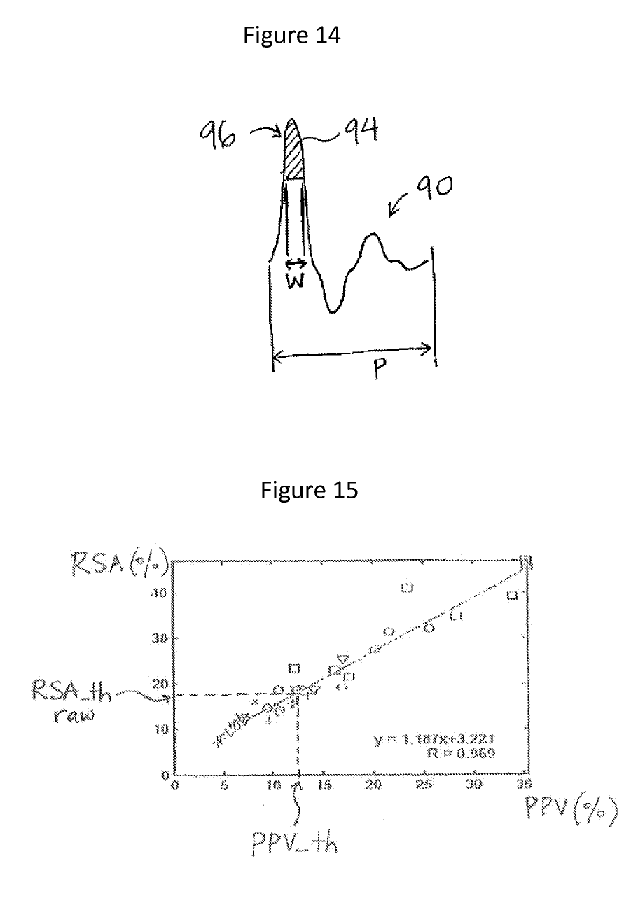

Additionally, as shown in FIG. 14, STT may be calculated by taking the derivative 90 of the PPG signal over one pulse period P, and measuring the area 94 under the fundamental pulse 96 of the derivative signal. For example, the area may be calculated over a defined time window or width W. The area describes a rise in the PPG upstroke, indicating a temporal extent of the rise. Thus, the area can be used as the STT metric, and can be calculated with each cardiac pulse to track STT over time and determine STTV. STT is also described in co-pending U.S. patent application Ser. No. 13/609,566, filed Sep. 11, 2012 (Publication No. 2014/0073962).

Another example of a non-invasive PPG-based FRP metric is respiratory sinus arrhythmia (RSA). RSA refers to the difference in frequency F between cardiac pulses within a respiratory cycle (see FIG. 1). In some patients, this frequency F between individual pulses changes with respiration. For example, the frequency may increase during inhalation and decrease during exhalation; that is, cardiac pulses are closer together during inhalation and spread more apart during exhalation. RSA can be calculated as follows:

RSA=(Rate_Max−Rate_Min)/Rate_Mean (10)

where Rate_Max is the maximum heart rate during a respiratory cycle, Rate_Min is the minimum heart rate during the cycle, and Rate_Mean is the average heart rate during the cycle. RSA may be derived from a PPG signal or other physiological signals such as EEG or EKG signal.

FIG. 15 shows a plot of RSA versus PPV, showing a strong linear correlation. The plotted data exhibited a relationship of RSA=m*PPV+c, where m=1.187 and c=3.221, with a Pearson correlation coefficient R=0.969. Accordingly, RSA may be used as an FRP herein, providing a non-invasive measure of fluid responsiveness. The fluid responsiveness threshold for PPV, PPV_th, corresponds to a raw RSA threshold, RSA_th_raw. The RSA_th_raw value may be used by clinicians to decide whether to provide or continue fluid therapy. Further, the numerical values of the two thresholds PPV_th and RSA_th_raw may not match; for example, PPV_th may be 13% while RSA_th_raw is 18%. As discussed herein, the RSA value may be scaled so that the RSA_th_adjusted value (not shown in FIG. 15) matches the PPV_th value.

Pre-processing of the PPG signal (or other physiologic signal) and post-processing of the calculated FRP number are described above. Other processing steps may include other modifications to the FRP value, such as correcting the FRP value for low perfusion. This technique is described in co-pending U.S. Patent Application No. 61/939,103. This technique includes adjusting the FRP value or formula when the PPG signal exhibits low perfusion. In an embodiment, such an adjustment is performed, and then the adjusted FRP value (adjusted for low perfusion) is scaled as described herein, to bring it into alignment with the PPV threshold.

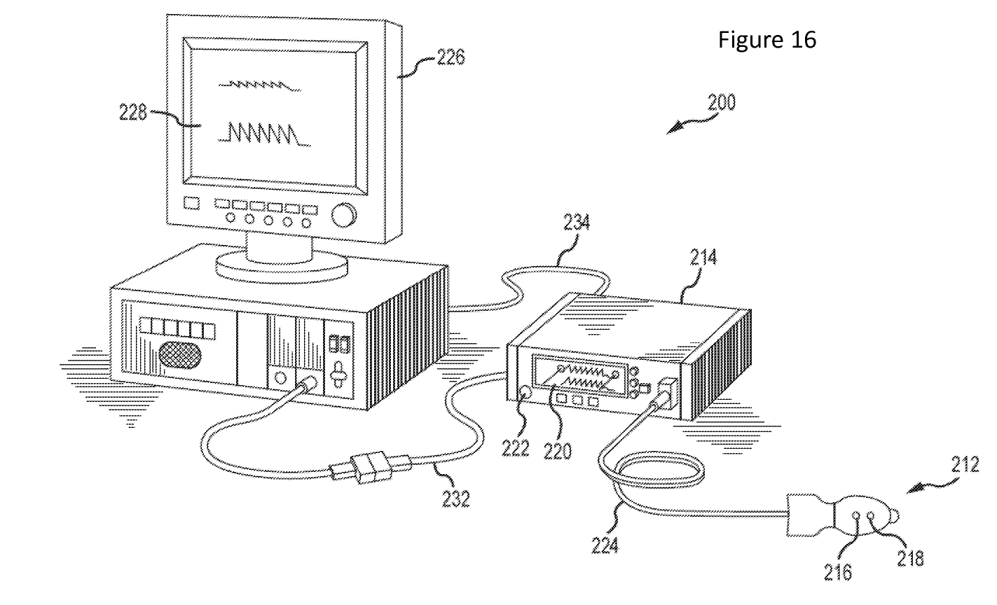

In an embodiment, the system and methods described above are implemented on a pulse oximeter. A pulse oximeter system 200 is illustrated in FIG. 16. The pulse oximeter non-invasively measures oxygen saturation of hemoglobin in arterial blood, by assessing a ratio of detected light at two wavelengths after illumination into a patient's tissue. For example, the oximeter may measure the intensity of light that is attenuated by the tissue and received at the light sensor, as a function of time. A signal representing light intensity or absorption versus time or a mathematical manipulation of this signal (e.g., a scaled version thereof, a log taken thereof, a scaled version of a log taken thereof, etc.) may be referred to as the photoplethysmograph (PPG) signal. The light intensity or the amount of light absorbed is then used to calculate the amount of a blood constituent (e.g., oxyhemoglobin) as well as the pulse rate and when each individual pulse occurs. To measure a constituent in the blood, the emitted light is selected to be of one or more wavelengths that are absorbed by blood in proportion to the blood constituent. For example, for determination of blood oxygen saturation (SpO₂) red and infrared wavelengths are used because highly oxygenated blood absorbs relatively less red light and more infrared light than blood with a lower oxygen saturation. By comparing the intensities of two wavelengths at different points in the pulse cycle, it is possible to estimate the blood oxygen saturation of hemoglobin in arterial blood.

Referring to FIG. 16, the pulse oximetry system 200 includes a sensor or probe 212 and a pulse oximetry monitor 214. The sensor 212 includes an emitter 216 configured to emit light at two or more wavelengths into a patient's tissue, and a detector 218 for detecting the light originally from the emitter 216 after passing through the tissue. The sensor 212 is connected via a cable 224 to the monitor 214, which includes a display 220 to display physiological data and speakers 222 to provide audible alarms. Calculations of physiological parameters from the PPG signal may take place on the sensor and/or the monitor. Optionally, the oximeter monitor 214 may be connected (via cable 232 or 234) to a multi-parameter patient monitor 226, which displays data from various medical devices on a display 228. The calculated FRP value may be displayed on the monitor 214, 226, or both.

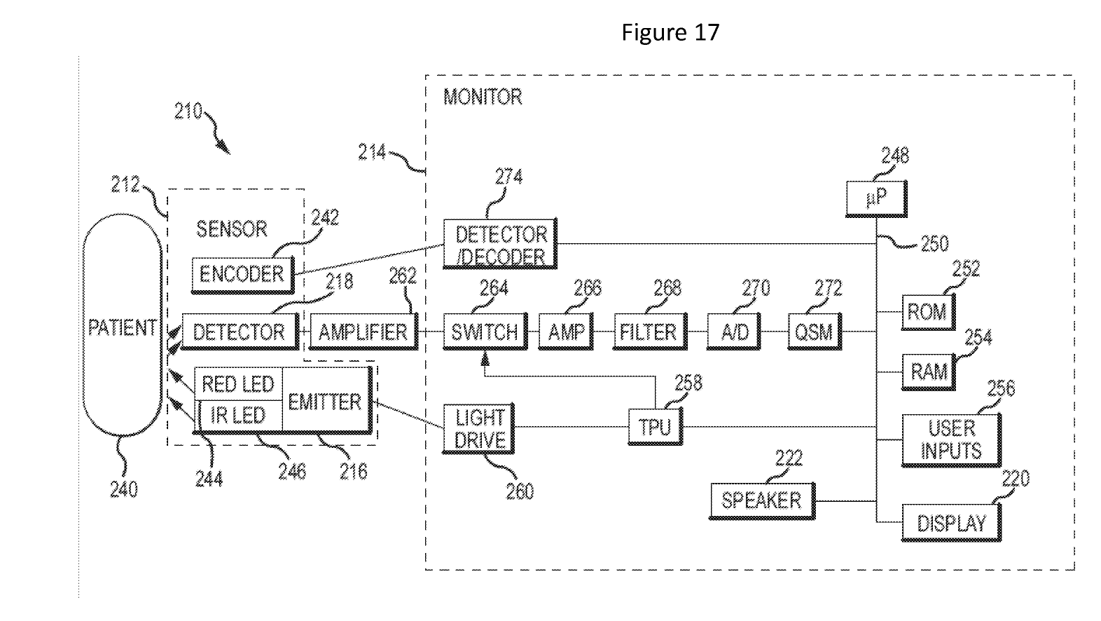

A simplified block diagram of the system 200 is shown in FIG. 17. Certain illustrative components of the sensor 212 and the monitor 214 are illustrated in FIG. 17. The sensor 212 includes the emitter 216, the detector 218, and an encoder 242. The emitter includes a RED light emitting light source 244, such as a light emitting diode (LED), and an infrared (IR) light emitting light source 246. In at least one embodiment, the RED wavelength may be between about 600 nm and about 700 nm, and the IR wavelength may be between about 800 nm and about 1000 nm. The detector detects light emitted or reflected from the patient's tissue 240, converts the received light into an electrical signal, and sends the signal to the monitor 214. The encoder 242 may contain information about the sensor 212, such as the type of sensor (for example, whether the sensor 212 is intended for placement on a forehead or digit) and the wavelengths of light emitted by the emitter 216. The information may be used by the monitor 214 to select appropriate algorithms, lookup tables and/or calibration coefficients stored in the monitor 214 for calculating the patient's physiological parameters.

The received signal from the detector 218 may be passed through an amplifier 266, a low pass filter 268, and an analog-to-digital converter 270. The digital data may then be stored in a queued serial module (QSM) 272 (or buffer) for later downloading to RAM 254 as QSM 272 fills up.

The monitor 214 includes a general-purpose microprocessor 248 connected to an internal bus 250. Also connected to the bus 250 are a read-only memory (ROM) 252, a random access memory (RAM) 254, user inputs 256 (such as patient information, alarm limits, etc), display 220, and speaker 222. The microprocessor 248 determines the patient's physiological parameters, such as SpO₂, respiration rate, respiratory effort, and pulse rate, using various algorithms and/or look-up tables based on the value of the received signals and/or data corresponding to the light received by the detector 218. Information from the encoder 242 is transmitted to a decoder 274, which translates the information to enable the processor 248 to use appropriate thresholds, algorithms, or other information. A time processing unit (TPU) 258 provides timing control signals to a light drive circuitry 260, which controls when the emitter 216 is illuminated and multiplexed timing for the RED LED 244 and the IR LED 246. The TPU 258 may control the sampling of signals from the detector 218 through an amplifier 262 and a switching circuit 264.

It is to be understood that the above description is intended to be illustrative, and not restrictive. Many modifications may be apparent to those skilled in the art to adapt a particular situation or system to the teachings of the present invention, without departing from its scope. The scope of the disclosure should, therefore, be determined with reference to the appended claims, along with the full scope of equivalents to which such claims are entitled.

What is claimed is:

1. A medical monitor for monitoring a patient, comprising:
an input receiving a photoplethysmograph (PPG) signal representing light absorption by a patient's tissue;
a fluid responsiveness predictor (FRP) calculator programmed to calculate an FRP metric; and
a memory storing a relationship between the FRP metric and a pulse pressure variation (PPV) metric,
wherein the FRP metric is calculated based on a respiratory variation of the PPG signal and based on the relationship.

2. The monitor of claim 1, wherein the FRP metric comprises Delta POP.

3. The monitor of claim 1, wherein the FRP metric comprises slope transit time variation.

4. The monitor of claim 1, wherein the FRP metric comprises respiratory sinus arrhythmia.

5. The monitor of claim 1, wherein the relationship comprises a scaling factor applied to the FRP metric.

6. The monitor of claim 1, wherein the relationship comprises a shift applied to the FRP metric.

7. The monitor of claim 1, wherein the relationship comprises a mapping relationship mapping an FRP threshold value to a pulse pressure variation threshold value.

8. The monitor of claim 1, wherein the relationship is adjustable based on a user input or an update.

9. The monitor of claim 1, further comprising a display in communication with the FRP calculator to display the calculated FRP metric.

10. The monitor of claim 1, further comprising a pre-processor coupled to the input to process the PPG signal.

11. The monitor of claim 1, wherein the memory stores a plurality of relationships between the FRP metric and PPV, and wherein the FRP calculator is programmed to calculate the FRP metric based on a selected relationship from the plurality of relationships.

12. The monitor of claim 1, wherein the respiratory variation of the PPG signal comprises an amplitude modulation of the PPG signal.

13. A method for predicting a fluid responsiveness of a patient, comprising:
receiving a photoplethysmograph (PPG) signal responsive to light absorption by a patient's tissue;
identifying a respiratory-induced variation of the PPG signal;
storing a relationship between a fluid responsiveness metric and a pulse pressure variation;
determining a value of the fluid responsiveness metric based on the respiratory-induced variation and the relationship.

14. The method of claim 13, further comprising displaying the fluid responsiveness metric.

15. The method of claim 13, wherein the fluid responsiveness metric comprises Delta POP.

16. The method of claim 13, further comprising determining an updated relationship, storing the updated relationship, and determining the value of the fluid responsiveness metric based on the respiratory-induced variation and the updated relationship.

17. The method of claim 13, further comprising storing a plurality of relationships and selecting a relationship for the determining the value of the fluid responsiveness metric.

18. The method of claim 17, wherein selecting the relationship is based on a user input.

19. A medical monitor for monitoring vital signs of a patient, comprising:
an electrical input providing a photoplethysmography (PPG) signal responsive to light absorption by a patient's tissue;
a fluid responsiveness calculator programmed to calculate a Delta POP (DPOP) value based on a respiratory variation of the PPG signal;
a scaling unit operating on the DPOP value to provide a scaled DPOP based on a relationship between DPOP and pulse pressure variation; and
an output for providing the DPOP value or the scaled DPOP value to a display.
